(19) United States
(12) Patent Application Publication
(10) Pub. No.: US 2018/0168454 A1
(43) Pub. Date: Jun. 21, 2018

(54) DEVICE INCLUDING LIGHT SOURCE EMITTING PULSED LIGHT, LIGHT DETECTOR, AND PROCESSOR

(71) Applicant: Panasonic Intellectual Property Management Co., Ltd., Osaka (JP)

(72) Inventors: TAKAMASA ANDO, Osaka (JP); TERUHIRO SHIONO, Osaka (JP)

(21) Appl. No.: 15/834,041

(22) Filed: Dec. 6, 2017

(30) Foreign Application Priority Data
Dec. 15, 2016 (JP) 2016-243291

(51) Int. Cl. A61B 5/00

(52) U.S. Cl. CPC A61B 5/0075; A61B 5/0077; A61B 5/0082

(57) ABSTRACT

A device is used for measurement of an internal portion of an object and includes a light source that emits pulsed light with which the object is irradiated, a light detector that detects light which returns from the object in response to irradiation with the pulsed light, and a processor. The processor assesses temporal stability of a light amount of the light which returns from the object and is detected by the light detector.

### BACKGROUND

### 1. Technical Field

The present disclosure relates to a device that is used for measurement of an internal portion of an object.

### 2. Description of the Related Art

In the field of living body measurement, a method is used which irradiates an object with light and acquires internal information of the object from information of light which is transmitted through an internal portion of the object. In this method, surface reflection components that are reflection components from a surface of the object may become noise. As a method that removes the noise due to those surface reflection components and acquires only desired internal information, in the field of living body measurement, there is a method disclosed by Japanese Unexamined Patent Application Publication No. 11-164826, for example. Japanese Unexamined Patent Application Publication No. 11-164826 discloses a method in which a light source and a light detector are brought into tight contact with a measured site in a state where the light source and the light detector are separated at a regular interval for measurement.

### SUMMARY

In one general aspect, the techniques disclosed here feature a device that is used for measurement of an internal portion of an object, the device including: a light source that emits pulsed light with which the object is irradiated; a light detector that detects light which returns from the object in response to irradiation with the pulsed light; and a processor. The processor assesses temporal stability of a light amount of the light that returns from the object and is detected by the light detector.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a schematic diagram that illustrates an imaging device of a first embodiment and a situation in which the imaging device photographs an object;

FIG. 1B is a diagram that illustrates one example of a configuration of an image sensor;

FIG. 1C is a flowchart that illustrates an outline of an action by a control circuit;

FIG. 2 is a diagram that illustrates a waveform of a surface reflection component, a waveform of an internally scattered component, a waveform in which the surface reflection component and the internally scattered component are combined, and timings of OPEN and CLOSE of an electronic shutter;

FIG. 3 is a flowchart that illustrates an action of the imaging device in the first embodiment at a time before final measurement;

FIG. 4A illustrates one example of an assessment by a measurement environment assessment unit;

FIG. 4B illustrates one example of the assessment by the measurement environment assessment unit;

FIG. 4C illustrates one example of the assessment by the measurement environment assessment unit;

FIG. 4D illustrates one example of the assessment by the measurement environment assessment unit;

FIG. 5A is a diagram that illustrates one example of a display that displays a photographed image which is obtained by the imaging device and a detection region of the object;

FIG. 5B is a diagram that illustrates one example of a display that displays the photographed image which is obtained by the imaging device and the detection region of the object;

FIG. 5C is a diagram that illustrates the detection region at a time after a size and a position are adjusted;

FIG. 5D is a diagram that illustrates the detection region which is maximized by a region maximization function;

FIG. 5E is a diagram that illustrates plural detection regions on the photographed image;

FIG. 6A is a diagram that illustrates one example of an error message which is output to the display in a case where the detection region is assessed as not correct in the measurement environment assessment unit;

FIG. 6B is a diagram that illustrates additional lines which are indicated on the display in order to facilitate adjustment of the detection region of the object;

FIG. 6C is a diagram of an adjustment stage for adjusting the detection region by adjusting orientation and position of the imaging device;

FIG. 6D is a diagram that illustrates a fixing jig for fixing the object;

FIG. 7A is a diagram that illustrates a circumstance in which light amount adjustment is requested;

FIG. 7B is a diagram that illustrates a circumstance in which light amount adjustment is requested;

FIG. 7C is a diagram that illustrates the relationship among plural light emission pulses, optical signals thereof on a sensor, plural shutter timings, and charge storage timings in one frame;

FIG. 8A is a diagram that illustrates one example of an assessment in a signal stability assessment unit;

FIG. 8B is a diagram that illustrates one example of the assessment in the signal stability assessment unit;

FIG. 9 is a diagram that illustrates one example of an error message which is output to the display in a case where a signal is assessed as not stable by the signal stability assessment unit;

FIG. 10A is a schematic diagram that illustrates an imaging device of a second embodiment and a situation in which the imaging device photographs the object;

FIG. 10B is a flowchart that illustrates an action of the imaging device in the second embodiment during the final measurement;

FIG. 11A is a diagram that illustrates an example of an assessment in an abnormal value assessment unit;

FIG. 11B is a diagram that illustrates an example of an assessment in the abnormal value assessment unit;

FIG. 12A is a diagram that illustrates one example of an error message which is output to the display in a case where an abnormal value is assessed as occurring in the abnormal value assessment unit; and

FIG. 12B is a diagram that illustrates one example of an error message which is output to the display in a case where the abnormal value is assessed as occurring in the abnormal value assessment unit.

### DETAILED DESCRIPTION

However, in the method disclosed in Japanese Unexamined Patent Application Publication No. 11-164826, because a light detector is brought into tight contact with a measured site and a psychological or physical load on a subject is high, time is requested for mounting, and use for a long time is difficult.

The present disclosure includes aspects that are described in the following items, for example.

### [Item 1]

A device according to item 1 of the present disclosure is

a device that is used for measurement of an internal portion of an object, the device including:

a light source that emits pulsed light with which the object is irradiated;

a light detector that detects light which returns from the object in response to irradiation with the pulsed light; and

a processor.

The processor assesses temporal stability of a light amount of the light which returns from the object and is detected by the light detector.

### [Item 2]

In the device according to item 1,

the processor may assess the temporal stability by determining whether a temporal change of the light amount of the light which returns from the object and is detected by the light detector is within a criteria, and

when it is determined that the temporal change is within the criteria, the processor may generate information regarding the internal portion of the object based on a signal from the light detector.

### [Item 3]

In the device according to item 1 or 2,

the light detector may be an image sensor that converts the light which returns from the object into a signal charge and stores the signal charge, and

the processor may assess the temporal stability by assessing temporal stability of a storage amount of the signal charge in the image sensor.

### [Item 4]

In the device according to any of items 1 to 3,

the processor may further, before assessing the temporal stability:
assess whether an environment of the object is suitable for the measurement of the internal portion of the object, and
adjust a light amount of the pulsed light.

### [Item 5]

In the device according to item 4,

the processor may assess whether the environment of the object is suitable for the measurement by determining whether information regarding the environment of the object is within a criteria.

### [Item 6]

In the device according to item 5,

the processor may determine whether the information regarding the environment of the object is within the criteria by determining whether a position of a region that is used for the measurement of the internal portion of the object is present in a desired position of the object.

### [Item 7]

In the device according to item 5,

the processor may determine whether the information regarding the environment of the object is within the criteria by determining whether an amount of a disturbance light that enters the light detector from outside the object is within the criteria.

### [Item 8]

In the device according to item 4,

the processor may adjust the light amount of the pulsed light by adjusting a light emission frequency of the pulsed light per unit time.

### [Item 9]

In the device according to item 3,

the image sensor may acquire a first image of the object based on the signal charge, and

the processor may further decide a position of a region that is used for the measurement of the internal portion of the object in the first image.

### [Item 10]

In the device according to item 9,

the object may be a living body,

the region may be an inside of a specific site of the living body, and

the processor may further adjust a size of the region so as to maximize the region in the inside of the specific site.

### [Item 11]

The device according to item 9 or 10 may further include

a display, and

the display may display the first image and a second image that indicates the region while superimposing the second image on the first image.

### [Item 12]

In the device according to item 11,

the display may further display an additional line for deciding the position of the region while superimposing the additional line on the first image and the second image.

### [Item 13]

In the device according to any of items 1 to 12,

the processor may further assess whether an abnormal value occurs during the measurement of the internal portion of the object.

### [Item 14]

In the device according to item 3,

the image sensor may store the signal charge that corresponds to a component, which is scattered in the internal portion of the object, of the light which returns from the object.

### [Item 15]

In the device according to any of items 1 to 14,

the object may be a living body, and

the processor may generate information that indicates a blood flow change of the living body based on a signal from the light detector.

### [Item 16]

A method according to item 16 of the present disclosure is

a method that is used for measurement of an internal portion of an object, the method including:

irradiating the object with pulsed light;

detecting light which returns from the object by a light detector in response to irradiation with the pulsed light; and

assessing temporal stability of a light amount of the light which returns from the object and is detected by the light detector.

### [Item 17]

In the method according to item 16,

the light detector may be an image sensor that converts the light which returns from the object into a signal charge and stores the signal charge, and

in the assessing,

temporal stability of a storage amount of the signal charge in the image sensor may be assessed to assess the temporal stability of the light amount of the light which returns from the object and is detected by the light detector.

### [Item 18]

The method according to item 16 or 17 may further include:

assessing whether an environment of the object is suitable for the measurement of the internal portion of the object; and

adjusting a light amount of the pulsed light.

### [Item 19]

In the method according to any of items 16 to 18,

the object may be a living body, and

the method may further include generating information that indicates a blood flow change of the living body based on a signal from the light detector.

In the present disclosure, all or a part of any of circuit, unit, device, part, or portion, or all or a part of functional blocks in the block diagrams may be implemented as one or more of electronic circuits including, but not limited to, a semiconductor device, a semiconductor integrated circuit (IC), or a large scale integration (LSI). The LSI or IC can be integrated into one chip, or also can be a combination of plural chips. For example, functional blocks other than a memory may be integrated into one chip. The name used here is LSI or IC, but it may also be called system LSI, very large scale integration (VLSI), or ultra large scale integration (ULSI) depending on the degree of integration. A field programmable gate array (FPGA) that can be programmed after manufacturing an LSI or a reconfigurable logic device that allows reconfiguration of the connection or setup of circuit cells inside the LSI can be used for the same purpose.

Further, it is also possible that all or a part of the functions or operations of the circuit, unit, device, part, or portion are implemented by executing software. In such a case, the software is recorded on one or more non-transitory recording media such as a ROM, an optical disk, or a hard disk drive, and when the software is executed by a processor, the software causes the processor together with peripheral devices to execute the functions specified in the software. A system or apparatus may include such one or more non-transitory recording media on which the software is recorded and a processor together with necessary hardware devices such as an interface.

In one aspect of the present disclosure, internal information of an object may be measured in a state where contact is not made with the object and in a state where noise due to a reflection component from a surface of the object is suppressed. Further, in one aspect of the present disclosure, an object may stably be measured while error factors due to contactless measurement are omitted.

All the embodiments described in the following illustrate general or specific examples. Values, shapes, materials, configuration elements, arrangement positions of configuration elements, and so forth that are described in the following embodiments are examples and are not intended to limit the present disclosure. Further, the configuration elements that are not described in the independent claims which provide the most superordinate concepts among the configuration elements in the following embodiments will be described as arbitrary configuration elements.

Embodiments will hereinafter be described in detail with reference to drawings.

### First Embodiment

### [1. Imaging Device]

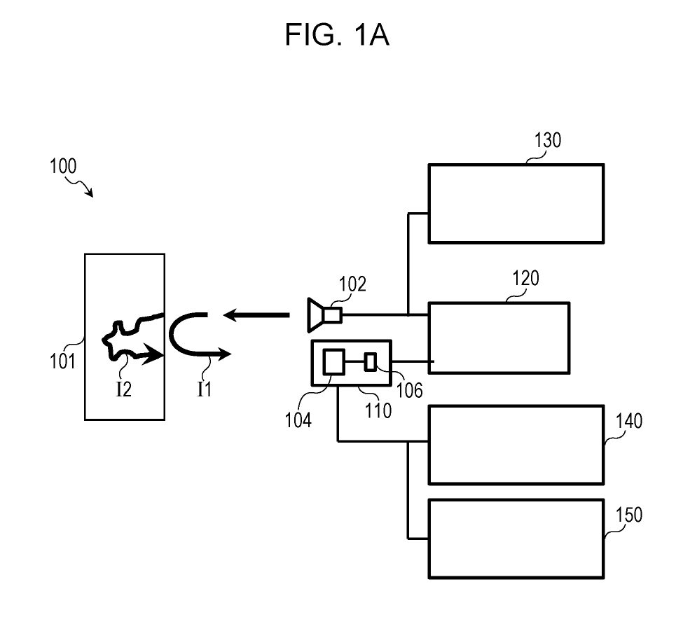

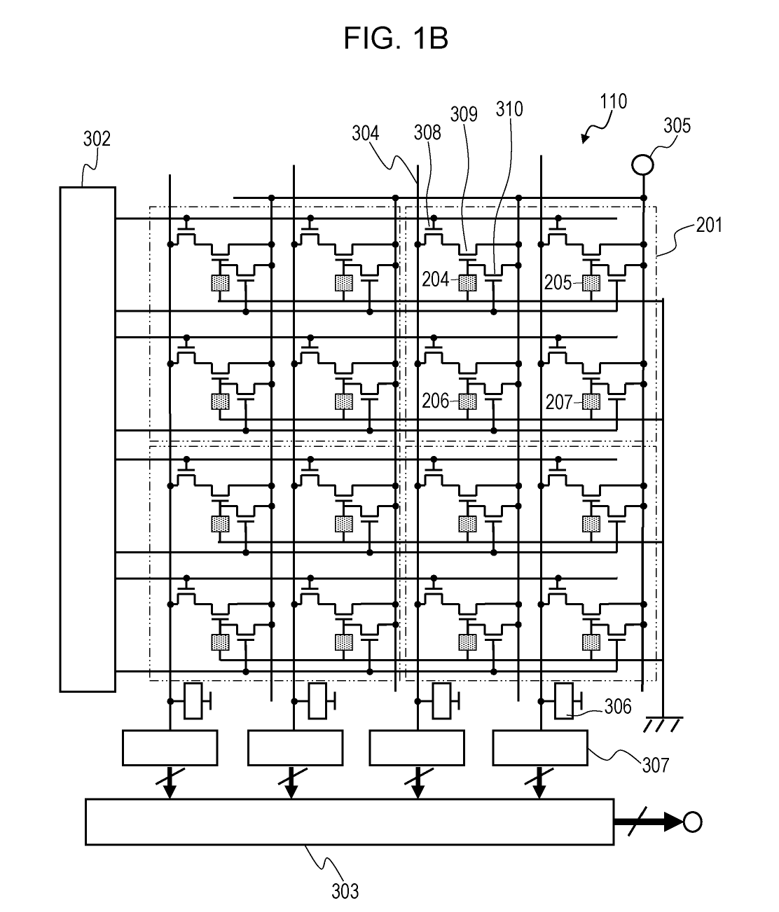

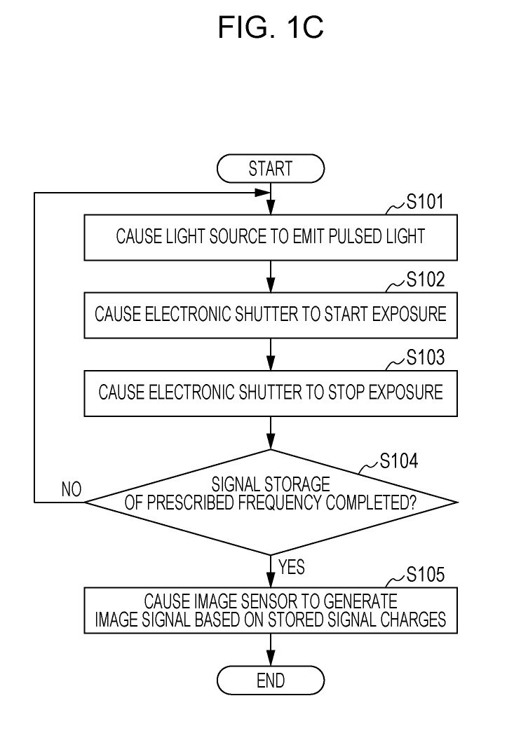

First, a configuration of an imaging device 100 according to a first embodiment will be described with reference to FIG. 1A to FIG. 3.

FIG. 1A is a schematic diagram that illustrates the imaging device 100 according to this embodiment. The imaging device 100 includes a light source 102, an image sensor 110 that includes a photoelectric conversion unit 104 and a charge storage unit 106, a control circuit 120, an emission light amount adjustment unit 130, a measurement environment assessment unit 140, and a signal stability assessment unit 150. The image sensor 110 is correspondent to a light detector. The emission light amount adjustment unit 130, the measurement environment assessment unit 140, and the signal stability assessment unit 150 are correspondent to a processor.

### [1-1. Light Source 102]

The light source 102 irradiates an object 101 with light. The light that is irradiated from the light source 102 and reaches the object 101 becomes a surface reflection component I1 that is a component which is reflected on a surface of the object 101 and an internally scattered component I2 that is a component which is one time reflected or scattered or multiply scattered in an internal portion of the object 101. The surface reflection component I1 includes three components of a direct reflection component, a diffused reflection component, and a scattered reflection component. The direct reflection component is a reflection component whose incident angle and reflection angle are equivalent. The diffused reflection component is a component that is reflected while being diffused by an uneven shape of the surface. The scattered reflection component is a component that is reflected while being scattered by an internal tissue in the vicinity of the surface. In a case where the object 101 is the forehead of a person, the scattered reflection component is a component that is reflected while being scattered by an internal portion of the epidermis. Hereinafter, in the present disclosure, a description will be made on an assumption that the surface reflection component I1 of the object 101 includes those three components. Further, a description will be made on an assumption that the internally scattered component I2 does not include the component that is reflected while being scattered by the internal tissue in the vicinity of the surface.

Traveling directions of the surface reflection component I1 and the internally scattered component I2 change due to reflection or scatter, and portions of the surface reflection component I1 and the internally scattered component I2 reach the image sensor 110. The light source 102 produces pulsed light plural times at prescribed time intervals or timings. A fall time of the pulsed light produced by the light source 102 may be close to zero, and the pulsed light is a rectangular wave, for example. In general, considering that the extension of the rear end of the internally scattered component I2 of the object 101 is 4 ns, the fall time may be 2 ns or lower, which is half the extension or lower, or may be 1 ns or lower. A rise time of the pulsed light produced by the light source 102 may be arbitrary. This is because a fall portion of the pulsed light along a time axis is used but a rise portion is not used in the measurement that uses the imaging device of the present disclosure and will be described later. The light source 102 is laser such as an LD in which the fall portion of the pulsed light is close to a right angle to the time axis and the time response characteristic is rapid, for example.

The wavelength of the pulsed light that is emitted from the light source 102 may be set to approximately 650 nm or more to approximately 950 nm or less, for example. This wavelength range is included in the wavelength range of red to near infrared rays. This wavelength region is a wavelength band in which light is easily transmitted to the internal portion of the object 101. Herein, a term of “light” will be used for not only visible light but also infrared rays.

Because the imaging device 100 of the present disclosure contactlessly measures the object 101, an influence on the retina is taken into consideration in a case where the object 101 is a person. Thus, class 1 of laser safety standards that are held by each country may be satisfied. In this case, the object 101 is irradiated with light with such a low illumination that the accessible emission limit (AEL) is below 1 mW. However, the light source 102 itself may not satisfy class 1. For example, it is sufficient that a diffusion plate, an ND filter, or the like is placed in front of the light source 102, light is diffused or attenuated, and class 1 of laser safety standards is thereby satisfied.

A streak camera in related art, which is disclosed in Japanese Unexamined Patent Application Publication No. 4-189349 and so forth, has been used for distinctively detecting information (for example, absorption coefficient and scattering coefficient) that is present in a different place in the depth direction of an internal portion of a living body. Accordingly, in order to perform measurement with desired spatial resolution, ultra-short pulsed light whose pulse width is femtoseconds or picoseconds has been used. On the other hand, the imaging device 100 of the present disclosure is used for distinctively detecting the internally scattered component I2 from the surface reflection component I1.

Accordingly, the pulsed light emitted by the light source 102 does not have to be the ultra-short pulsed light, and the pulse width is arbitrary. In a case where light is applied to the forehead to measure the brain blood flow, the light amount of the internally scattered component I2 becomes a very small amount such as one several-thousandth to one several-ten-thousandth compared to the light amount of the surface reflection component I1. In addition, taking into consideration laser safety standards, the light amount of the light with which irradiation may be performed is small, and detection of the internally scattered component I2 becomes difficult. Accordingly, the light source 102 produces pulsed light with a comparatively large pulse width, the integrated amount of the internally scattered component with a time delay is thereby increased, the detected light amount is increased, and the SN ratio may thereby be improved.

The light source 102 emits the pulsed light with a pulse width of 3 ns or more, for example. Alternatively, the light source 102 may emit the pulsed light with a pulse width of 5 ns or more or further 10 ns or more. Meanwhile, because unused light increases and is wasted in a case where the pulse width is too large, the light source 102 produces the pulsed light with a pulse width of 50 ns or less, for example. Alternatively, the light source 102 may emit the pulsed light with a pulse width of 30 ns or less or further 20 ns or less.

Note that an irradiation pattern of the light source 102 may have a uniform intensity distribution in an irradiation region. A method disclosed in Japanese Unexamined Patent Application Publication No. 11-164826 and so forth has to perform discrete light irradiation because a detector is separated from a light source by 3 cm and the surface reflection component I1 is spatially reduced. On the other hand, the imaging device 100 of the present disclosure uses a method in which the surface reflection component I1 is temporally separated and reduced. Thus, the internally scattered component I2 may also be detected on the object 101 immediately under an irradiation point. In order to enhance measurement resolution, irradiation may be performed spatially all over the object 101.

### [1-2. Image Sensor 110]

The image sensor 110 receives the light that is emitted from the light source 102 and is reflected by the object 101. The image sensor 110 has plural pixels that are two-dimensionally arranged and acquires two-dimensional information of the object 101 at a time. The image sensor 110 is a CCD image sensor or a CMOS image sensor, for example.

The image sensor 110 has an electronic shutter. The electronic shutter is a circuit that controls one signal storage period in which received light is converted into effective electrical signals and stored, that is, a shutter width which is a length of an exposure period and a shutter timing which is a time from a finish of one exposure period to a start of a next exposure period. Hereinafter, a description may be made while a state where the electronic shutter performs exposure is referred to as “OPEN (open state)” and a state where the electronic shutter stops exposure is referred to as “CLOSE (close state)”.

The image sensor 110 may adjust the shutter timing by the electronic shutter in subnano-seconds, for example, 30 ps to 1 ns. A TOF camera in related art which is intended to perform distance measurement detects the whole light that is the pulsed light which is emitted by the light source 102, is reflected by a photographed object, and is returned in order to correct an influence of brightness of the photographed object. Accordingly, in the TOF camera in related art, the shutter width has to be larger than the pulse width of light. On the other hand, because the imaging device 100 of this embodiment does not have to correct the light amount of the photographed object, the shutter width does not have to be larger than the pulse width and is approximately 1 to 30 ns, for example. In the imaging device 100 of this embodiment, the shutter width may be shrunk, and dark current included in detection signals may thus be reduced.

In a case where the object 101 is the forehead of a person and information such as the brain blood flow is detected, the light attenuation rate in an internal portion is very high and is approximately one millionth. Thus, to detect the internally scattered component I2, the light amount may be insufficient with only one pulse irradiation. Irradiation of class 1 of laser safety standards provides a very minute light amount. In this case, the light source 102 emits the pulsed light plural times, the image sensor 110 performs exposure plural times by the electronic shutter in response to that, the detection signals are thereby integrated, and sensitivity is improved.

In the following, a configuration example of the image sensor 110 will be described.

The image sensor 110 has pixels as plural light detection cells that are two-dimensionally arranged on an imaging surface. Each of the pixels has a light-receiving element (for example, a photodiode).

FIG. 1B is a diagram that illustrates one example of a configuration of the image sensor 110. In FIG. 1B, the region surrounded by a frame of two-dot chain lines is correspondent to one pixel 201. The pixel 201 includes one photodiode. Although FIG. 1B illustrates only four pixels that are aligned in two rows and two columns, further many pixels are actually arranged. The pixel 201 includes the photodiode, a source follower transistor 309, a row-select transistor 308, and a reset transistor 310. Each transistor is a field effect transistor that is formed on a semiconductor substrate, for example. However, the transistor is not limited to this.

As illustrated in FIG. 1B, one (typically, source) of an input terminal and an output terminal of the source follower transistor 309 is connected with one (typically, drain) of an input terminal and an output terminal of the row-select transistor 308. A gate that is a control terminal of the source follower transistor 309 is connected with the photodiode. A signal charge (electron hole or electron) that is generated by the photodiode is stored in floating diffusion layers 204, 205, 206, and 207 as charge storage units that are charge storage nodes between the photodiode and the source follower transistors 309.

Although not illustrated in FIG. 1B, a switch may be provided between the photodiode and the floating diffusion layers 204, 205, 206, and 207. This switch switches conduction states between the photodiode and the floating diffusion layers 204, 205, 206, and 207 in response to a control signal from the control circuit 120. Consequently, start and stop of storage of the signal charges in the floating diffusion layers 204, 205, 206, and 207 are controlled. The electronic shutter in this embodiment has a mechanism for such exposure control.

The signal charges stored in the floating diffusion layer 204, 205, 206, and 207 are read out by turning ON a gate of the row-select transistor 308 by a row-select circuit 302. Here, the current that flows from a source follower power source 305 to the source follower transistors 309 and a source follower load 306 is amplified in accordance with the signal potential of the floating diffusion layers 204, 205, 206, and 207. An analog signal due to this current that is read out from a vertical signal line 304 is converted into digital signal data by an analog-digital (AD) conversion circuit 307 that is connected for each column. The digital signal data are read out for each column by a column-select circuit 303 and are output from the image sensor 110. The row-select circuit 302 and the column-select circuit 303 perform a read-out for one row and thereafter perform the read-out for the next row. Similarly for the following rows, information of the signal charges of the floating diffusion layers in all the rows are read out. The control circuit 120 reads out all the signal charges, thereafter turns ON a gate of the reset transistor 310, and thereby resets all the floating diffusion layers. Consequently, imaging for one frame is completed. Similarly for the other frames, high-speed imaging for the frame is repeated, and a series of imaging for the frames by the image sensor 110 is ended.

In this embodiment, an example of the image sensor 110 of a CMOS type is described. However, the image sensor 110 may be a CCD type, a single photon counting type element, or an amplifying type image sensor (EMCCD or ICCD).

### [1-3. Control Circuit 120]

The control circuit 120 adjusts the time difference between a light emission timing of the pulsed light of the light source 102 and the shutter timing of the image sensor 110. Hereinafter, the time difference may be referred to as “phase” or “phase delay”. “Light emission timing” of the light source 102 is a time when a rise of the pulsed light emitted by the light source 102 starts. The control circuit 120 may adjust the phase by changing the light emission timing or may adjust the phase by changing the shutter timing.

The control circuit 120 may be configured to remove an offset component from a signal detected by the light-receiving element of the image sensor 110. The offset component is a signal component due to sunlight, ambient light such as a fluorescent lamp, or disturbance light. In a state where the light source 102 does not emit light, that is, a state where driving of the light source 102 is turned OFF, the image sensor 110 detects the signal, and the offset component due to the ambient light or the disturbance light is thereby estimated.

The control circuit 120 may be an integrated circuit that has a processor such as a central processing unit (CPU) or a microcomputer and a memory, for example. The control circuit 120 executes a program recorded in the memory, for example, and thereby performs adjustment of the light emission timing and the shutter timing, estimation of the offset component, removal of the offset component, and so forth. Note that the control circuit 120 may include a computation circuit that performs a computation process such as image processing. Such a computation circuit may be realized by a combination of a digital signal processor (DSP), a programmable logic device (PLD) such as a field programmable gate array (FPGA), or a central processing unit (CPU) or a graphics processing unit (GPU), and a computer program, for example. Note that the control circuit 120 and the computation circuit may be one assembled circuit or may be separated individual circuits.

FIG. 1C is a flowchart that illustrates an outline of an action by the control circuit 120. Although details will be described later, the control circuit 120 generally executes the action illustrated in FIG. 1C. The control circuit 120 first causes the light source 102 to emit the pulsed light for a prescribed time (step S101). Here, the electronic shutter of the image sensor 110 is in a state where exposure is stopped. The control circuit 120 causes the electronic shutter to stop exposure until a period in which a portion of the pulsed light is reflected by the surface of the object 101 and reaches the image sensor 110 is completed. Next, the control circuit 120 causes the electronic shutter to start exposure at a timing when the other portion of the pulsed light is scattered in the internal portion of the object 101 and reaches the image sensor 110 (step S102). After a prescribed time elapses, the control circuit 120 causes the electronic shutter to stop exposure (step S103). Then, the control circuit 120 assesses whether or not the frequency of execution of the above signal storage reaches a prescribed frequency (step S104). In a case where the assessment is No, step S101 to step S103 are repeated until the assessment becomes Yes. In a case where the assessment is Yes in step S104, the control circuit 120 causes the image sensor 110 to generate and output signals that indicate an image based on the signal charges stored in the floating diffusion layers (step S105).

The above action enables a light component that is scattered in an internal portion of a measured object to be detected with high sensitivity. Note that light emission and exposure do not necessarily have to be performed plural times but are performed as necessary.

### [1-4. Other Matters]

The imaging device 100 may include an image formation optical system that forms a two-dimensional image of the object 101 on a light-receiving surface of the image sensor 110. An optical axis of the image formation optical system is substantially orthogonal to the light-receiving surface of the image sensor 110. The image formation optical system may include a zoom lens. In a case where the position of the zoom lens changes, the magnification ratio of the two-dimensional image of the object 101 is varied, and the resolution of the two-dimensional image on the image sensor 110 changes. Accordingly, it becomes possible to perform a detailed observation by magnifying a region to be measured even in a case where the distance to the object 101 is far.

Further, the imaging device 100 may include a band pass filter, which causes only the light in the wavelength band of the light emitted from the light source 102 or in the vicinity of the wavelength band to pass, between the object 101 and the image sensor 110. Consequently, the influence of a disturbance component such as the ambient light may be reduced. The band pass filter is configured with a multi-layer film filter or an absorption filter. The bandwidth of the band pass filter may have a width of approximately 20 to 100 nm in consideration of the band shift in accordance with the temperature of the light source 102 and the oblique incidence on the filter.

Further, the imaging device 100 may include respective polarizing plates between the light source 102 and the object 101 and between the image sensor 110 and the object 101. In this case, the polarizing directions of the polarizing plate arranged on the light source 102 side and the polarizing plate arranged on the image sensor side are in a crossed Nicols relationship. Consequently, a regular reflection component (a component whose incident angle and reflection angle are the same) of the surface reflection component I1 of the object 101 may be inhibited from reaching the image sensor 110. That is, the light amount of the surface reflection component I1 that reaches the image sensor 110 may be reduced.

### [2. Action]

The imaging device 100 of the present disclosure distinctively detects the internally scattered component I2 from the surface reflection component I1. In a case where the object 101 is the forehead of a person, the signal intensity of the internally scattered component I2 to be detected becomes very low. As described earlier, this is because irradiation is performed with the light with a very small light amount that satisfies laser safety standards and in addition the scatter and absorption of the light by the scalp, brain-cerebrospinal fluid, skull, gray matter, white matter, and blood flow are large. In addition, the change in the signal intensity due to the change in the blood flow rate or in components in the blood flow in a brain activity is correspondent to further one several-tenth magnitude and is very small. Accordingly, photographing is performed while entrance of the surface reflection component I1 that is as several thousand to several ten thousand times intense as the signal component to be detected is avoided as much as possible.

In the following, an action of the imaging device 100 in this embodiment will be described.

As illustrated in FIG. 1A, in a case where the light source 102 irradiates the object 101 with the pulsed light, the surface reflection component I1 and the internally scattered component I2 are produced. Portions of the surface reflection component I1 and the internally scattered component I2 reach the image sensor 110. Because the internally scattered component I2 passes through the internal portion of the object 101 between emission from the light source 102 and reaching the image sensor 110, the optical path length becomes long compared to the surface reflection component I1. Accordingly, as for the time to reach the image sensor 110, the internally scattered component I2 is averagely delayed compared to the surface reflection component I1.

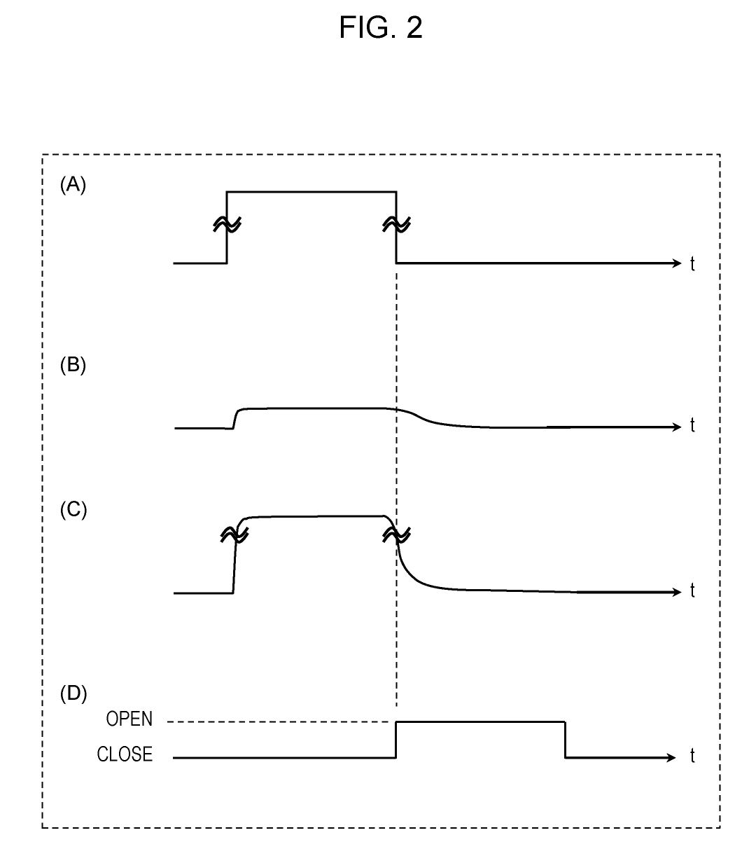

FIG. 2 is a diagram that represents optical signals in which a rectangular pulsed light is emitted from the light source 102 and the light reflected by the object 101 reaches the image sensor 110. In FIG. 2, a signal A indicates the waveform of the surface reflection component I1. A signal B indicates the waveform of the internally scattered component I2. A signal C indicates the waveform in which the surface reflection component I1 and the internally scattered component I2 are combined. A signal D indicates timings of OPEN and CLOSE of the electronic shutter. The horizontal axis represents time, and the vertical axis represents the light intensities in the signals A to C and represents a state of OPEN or CLOSE of the electronic shutter in the signal D.

As indicated by the signal A, the surface reflection component I1 maintains a rectangular shape. Meanwhile, as indicated by the signal B, because the internally scattered component I2 is the sum of beams of light that get through various optical path lengths, the internally scattered component I2 exhibits a characteristic that the fall time is longer than the surface reflection component I1 at a rear end of the pulsed light. In order to enhance the ratio of the internally scattered component I2 and extract the internally scattered component I2 from the signal C, as indicated by the signal D, the electronic shutter may start exposure after the rear end of the surface reflection component I1 (when the surface reflection component I1 falls or after that). This shutter timing is adjusted by the control circuit 120. As described above, because it is sufficient that the imaging device 100 of the present disclosure may distinctively detect the internally scattered component I2 from the surface reflection component I1, a light emission pulse width and the shutter width are arbitrary. Accordingly, the imaging device 100 may be realized by a simple configuration differently from a method that uses the streak camera in related art, and the cost may considerably be lowered.

As it may be understood from the signal A in FIG. 2, the rear end of the surface reflection component I1 falls vertically. In other words, the time between the start of fall of the surface reflection component I1 and the finish is zero. However, in reality, the pulsed light itself of irradiation by the light source 102 may not be perfectly vertical, fine unevenness may be present on the surface of the object 101, and the rear end of the surface reflection component I1 may not vertically fall due to scatter in the epidermis. Further, because the object 101 is often an opaque physical body in general, the light amount of the surface reflection component I1 is much larger than the internally scattered component I2. Accordingly, even in a case where the rear end of the surface reflection component I1 slightly projects from the vertical fall position, the internally scattered component I2 is covered, and a problem occurs. Further, due to a time delay accompanying electron transfer during a readout period of the electronic shutter, an idealistic binary readout as indicated by the signal D in FIG. 2 may not be realized. Accordingly, the control circuit 120 may slightly delay the shutter timing of the electronic shutter with respect to the time immediately after the fall of the surface reflection component I1. For example, in view of the accuracy of the electronic shutter, the shutter timing of the electronic shutter may be delayed by 1 ns or more with respect to the time immediately after the fall of the surface reflection component I1. Note that instead of adjusting the shutter timing of the electronic shutter, the control circuit 120 may adjust the light emission timing of the light source 102. The control circuit 120 may adjust the time difference between the shutter timing of the electronic shutter and the light emission timing of the light source 102. Note that in a case where the change in the blood flow rate or in the components in the blood flow in the brain activity is contactlessly measured and where the shutter timing is delayed too much, the internally scattered component I2 that is originally small further decreases. Thus, the shutter timing may be retained in the vicinity of the rear end of the surface reflection component I1. Because the time delay due to scatter in the object 101 is 4 ns, the maximum delay amount of the shutter timing is approximately 4 ns.

The light source 102 emits the pulsed light plural times, exposure is performed plural times at the shutter timing in the same phase as each pulsed light, and the detected light amount of the internally scattered component I2 may thereby be amplified.

Note that instead of arranging the band pass filter between the object 101 and the image sensor 110 or in addition to that, the control circuit 120 may perform photographing in the same exposure time in a state where the light source 102 is not caused to emit light and thereby estimate the offset component. The estimated offset component is removed as a difference from the signal detected by the light-receiving element of the image sensor 110. Consequently, a dark current component that occurs on the image sensor 110 may be removed.

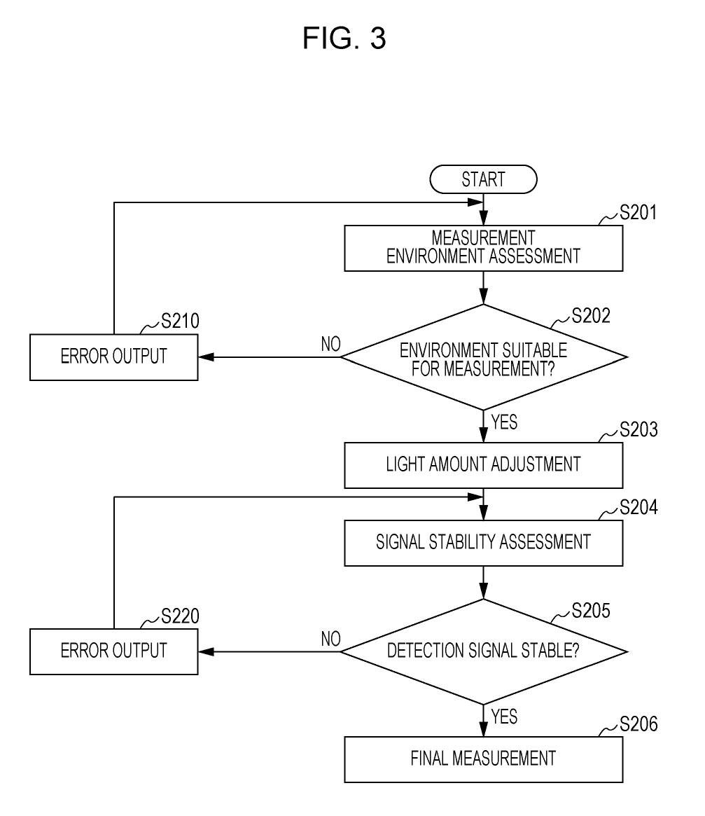

FIG. 3 is a flowchart that illustrates an action of the imaging device 100 in the first embodiment at a time before final measurement. After a start, the imaging device 100 uses the measurement environment assessment unit 140 to conduct a confirmation of whether or not the environment of the object 101 is in a state suitable for measurement (step S201). As a result of the confirmation of the measurement environment, in a case where the environment of the object 101 is assessed as not in the state suitable for the measurement (No in step S202), an error is output (step S210). In a case where the error is output, a measurement environment confirmation is again conducted after the error is handled. In a case where the environment is assessed as suitable for the measurement (Yes in step S202), light amount adjustment is thereafter conducted by the emission light amount adjustment unit 130 (step S203). In addition, after the light amount adjustment is completed, the stability of the detection signal is measured by the signal stability assessment unit 150 (step S204). In a case where the detection signal is assessed as not stable (No in step S205), an error is output (step S220). In a case where the error is output, signal stability measurement is again conducted after the error is handled. In a case where the detection signal is assessed as stable (Yes in step S205), the final measurement is started (step S206). The action is conducted in this order, and the measurement of the blood flow change of the living body may thereby be conducted efficiently, correctly, contactlessly, and highly accurately. For example, in a case where a signal stability assessment is conducted before a measurement environment assessment, the signal stability assessment unit 150 mistakenly determines that the signal is stable, hypothetically, even in a case where the imaging device 100 does not cover the object 101 but is photographing another stationary physical body, and the action progresses to the next step. Further, also in a case where emission light amount adjustment is conducted before the measurement environment assessment, the light amount is mistakenly adjusted in a case where another thing than the object 101 is photographed for a similar reason. Further, in a case where the signal stability measurement is conducted before the light amount adjustment, the SN of detection data of the imaging device 100 is lowered or saturated in a case where the light amount is too low or too high. Accordingly, as illustrated in FIG. 3, conducting the measurement environment assessment, the emission light amount adjustment, and the signal stability assessment in this order is optimal for living body measurement by using the imaging device 100 of the present disclosure.

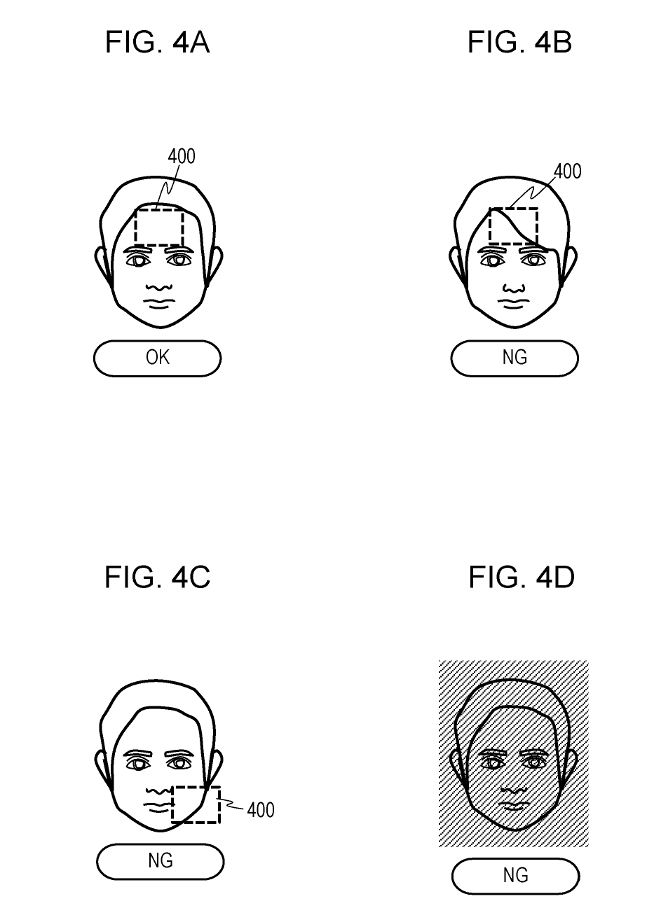

In the following, details of each function in a sequence in FIG. 3 will sequentially be described. FIG. 4A to FIG. 4D illustrate one example of an assessment by the measurement environment assessment unit 140. The measurement environment assessment unit 140 has a function to confirm whether a detection region 400 is present in a desired position of the object 101 and a disturbance error factor that influences the measurement is not present. For example, in a case where it is desired to observe the brain blood flow change of the frontal lobe by using the change in oxyhemoglobin and deoxyhemoglobin, the forehead is photographed as the object 101. Here, as in FIG. 4A, in a case where no other thing than the forehead appears in the detection region 400, the measurement environment assessment unit 140 assesses the environment as suitable for the measurement. However, in a case where other things than the forehead such as hair and a headband are included in the detection region 400 as in FIG. 4B or in a case where the detection region 400 is different from a place to be measured as in FIG. 4C, the measurement environment assessment unit 140 assesses the environment as not suitable for the measurement and outputs the error. Further, as in FIG. 4D, the disturbance light may enter. Whether the disturbance light enters may be determined by adding a mode for performing signal acquisition by the shutter without causing the light source 102 to emit the pulsed light and by confirming the pixel values of the offset component that is correspondent to the disturbance light. The disturbance light is light that includes near infrared rays at 750 to 850 nm which is close to the wavelength of a light source of irradiation, and room illumination such as an incandescent light bulb, halogen light, and xenon light in addition to sunlight may be factors. The slight disturbance light is removed by performing a difference computation process of the offset component that is estimated by performing a shutter action while irradiation with the light source 102 by the imaging device 100 is turned OFF. However, in a case where the offset component is excessively much, a dynamic range of the photodiode is lowered. Accordingly, for example, in a case where the offset component occupies half the dynamic range, the measurement environment assessment unit 140 assesses the environment as not suitable for the measurement.

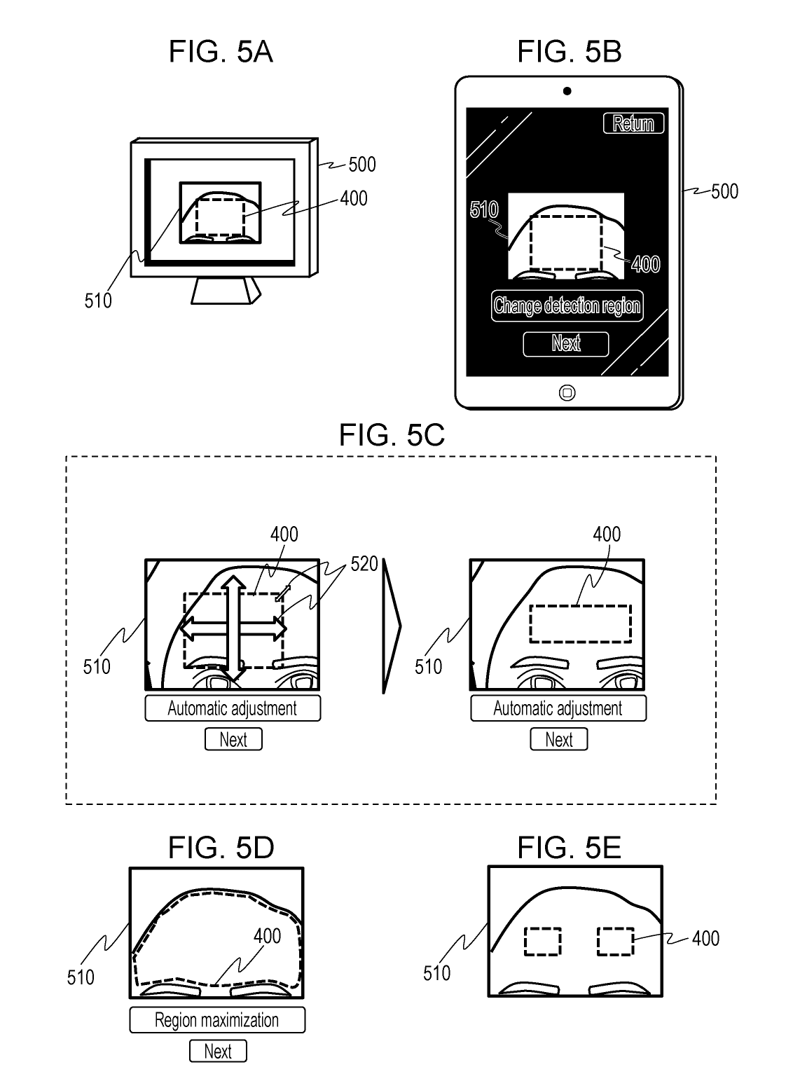

As in FIG. 5A, because the imaging device 100 also includes a function as a camera that photographs the face of the subject, the imaging device 100 displays a camera image on a display 500 such that the subject and an examiner may recognize whether the environment is an environment in which the measurement may be performed. Here, the detection region 400 is displayed while being superimposed on a photographed image 510. In a case where no masking object appears in the photographed image 510, the detection region 400 is magnified and may thereby be caused to match a whole region of the photographed image 510. In such a manner, the pixels of the image sensor of the imaging device 100 may be used efficiently, and the measurement with higher resolution may be realized. Further, as in FIG. 5B, in a case where a tablet or a smartphone is wirelessly connected as the display 500, more casual measurement may be realized anytime and anywhere such as a home or a visit destination.

In a case where another thing than a thing to be measured enters the initial detection region 400 as in FIG. 5C, a user may manually change the detection region 400. A position adjustment icon 520 is displayed on the photographed image 510, and the position and size of the detection region 400 may be changed by a drag operation or an input of coordinates. In a case where the forehead of the subject is small and the initial detection region 400 includes hair or the eyebrows, the detection region 400 is shrunk in accordance with the size of the forehead of the subject. Further, the measurement is performed while feature amounts of the eyes, eyebrows, nose, and the like are included in the region of the photographed image 510. Accordingly, an Automatic adjustment button is pressed, and thereby the detection region 400 is automatically set to a prescribed region of the forehead by face recognition computation. In a case where the masking object such as hair masks the forehead or the feature amounts are not correctly detected, an error that indicates that the detection region 400 may not be set is returned. Further, in a case where region maximization is turned ON by automatic adjustment, a portion in which the forehead is exposed is detected by image processing as in FIG. 5D, and the whole forehead may thereby be set as the detection region 400. In such a manner, a GUI for setting the detection region 400 is used, and it thereby becomes possible to perform adjustment such that two-dimensional distribution of the brain blood flow may be acquired correctly and easily or acquired maximally from the whole forehead.

Further, plural detection regions 400 may be provided as in FIG. 5E. A screen is tapped in order to increase the detection region 400. To delete the detection region 400, the detection region 400 to be deleted is long-tapped. Plural detection regions 400 are provided in specific sections, and evaluation that is specialized for the site of a focused brain activity thereby becomes possible. The load and transfer amount in data processing may be reduced because the data processing is only for information of a specific site.

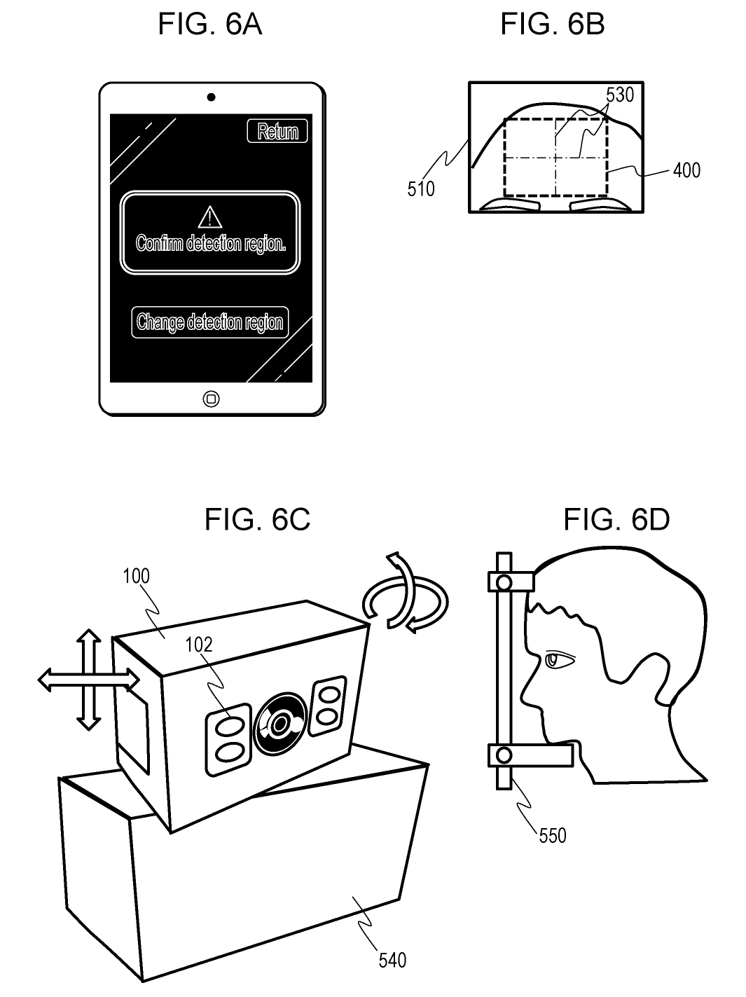

In a case where an attempt is made to start the measurement in a state where other things than the measured object such as hair and the eyebrows are included in the detection region 400, an error which advises a confirmation of whether the detection region 400 is correct is output by characters, voice, error sound, and so forth as in FIG. 6A. A determination whether other things than the measured object are included is realized by image processing by using an image acquired by the imaging device 100. For example, in a case where a local and excessive change in the contrast is seen in the intensity distribution in the detection region 400, a determination is made that another thing than the measured object enters. The excessive change in the contrast is a case where the pixel values change by, for example, 20% or more around the pixel of interest. The change in the contrast may easily be detected by using edge detection filters by Sobel, Laplacian, Canny, and so forth. Further, as another method, discrimination by pattern matching of feature amounts of disturbance factors or machine learning may be used. In a case where the forehead is detected, the disturbance factors are hair, the eyebrows, and so forth and are predictable to some extent. Thus, even a method that uses learning does not request very large data for prior learning and is thus easy to be realized. Note that an assessment subsequent to an exception process and smoothing may be added such that fine changes in the contrast such as moles and spots may be ignored.

In a case where the error of FIG. 6A is output, the detection region 400 is changed on the screen. In this case, manual or automatic adjustment of the detection region 400 is performed. Further, in a case where the region of the photographed image 510 is excessively displaced from a desired position and the detection region 400 may not be changed on the screen in a software manner, the subject himself/herself moves while confirming the display 500 and thereby sets the detection region 400 to the desired position. Here, as in FIG. 6B, it is desirable to display additional lines 530 on the display 500 such that the subject easily understands which position in the detection region 400 with respect to left, right, up, and down he/she is in. Based on the additional lines 530, adjustment between the center of the detection region 400 and the center of the forehead of the subject may be smoothly performed. In a case where the subject himself/herself performs adjustment while watching the display 500, it is desirable to display a mirror image that is a left-right inverted image as the photographed image 510 for facilitating adjustment. Further, the examiner may change the angle and position of the imaging device 100 while confirming the display 500 and may thereby adjust the detection region 400. As in FIG. 6C, an adjustment stage 540 for adjustment in x, y, and z directions and of inclinations (pan, tilt, and roll) is mounted on the imaging device 100, and the orientation of the imaging device 100 may be adjusted such that light irradiation and camera detection may be performed for the forehead of the subject. In addition, as in FIG. 6D, the subject is fixed by a fixing jig 550 for the chin and head of the subject, and the measurement in which a movement influence error is further reduced may thereby be performed. The examiner moves the imaging device 100 and performs adjustment, the load on the subject may thereby be reduced compared to a case where the subject himself/herself performs adjustment, and a psychological noise influence on acquired brain blood flow information may also be lowered.

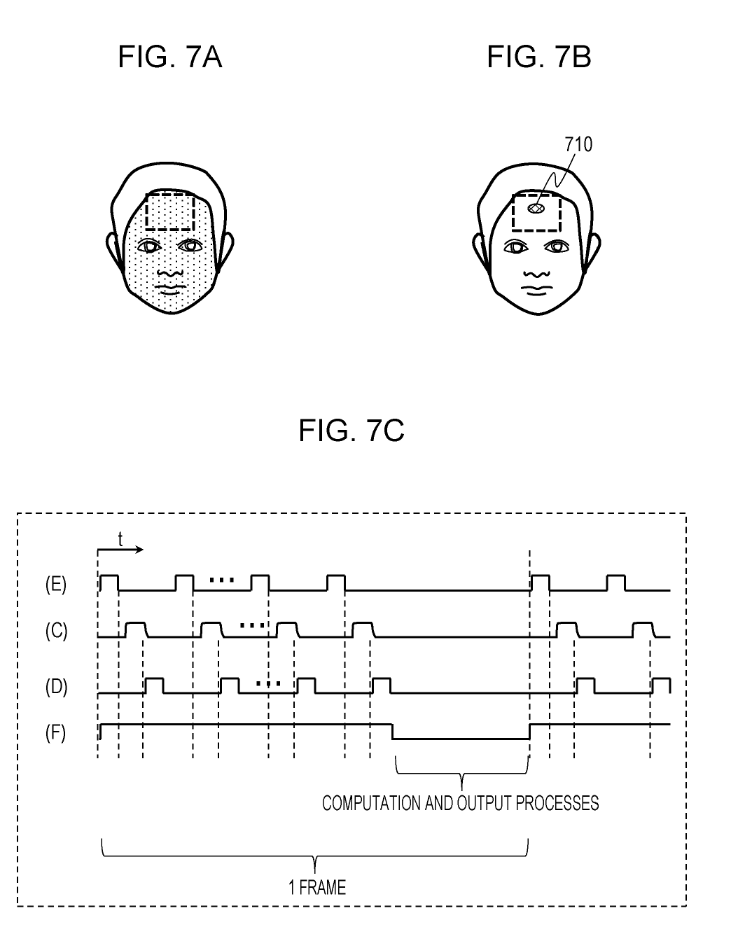

As illustrated in FIG. 7A and FIG. 7B, the brightness of the photographed image 510 that is detected by the imaging device 100 changes depending on the difference in the object 101. This is due to the color of the skin of the object 101, that is, the difference in the light absorption degree of a melanin pigment. In a case where the object 101 is too bright, the photographed image 510 is saturated, and the measurement may not be performed. The object 101 that is too dark is not desirable because the SN of the detected light amount is influenced. Accordingly, the emission light amount adjustment unit 130 adjusts the light amount of the light source 102 in accordance with the brightness of the object 101. Further, surface reflectance and diffusivity are different among individuals in accordance with the sweating state and skin shape of the object 101. As illustrated in FIG. 7B, in a case where shininess 710 is seen on the object 101, the emission light amount adjustment unit 130 adjusts the light amount so as to avoid saturation.

Because the imaging device 100 detects the very slight light that reaches the inside of the brain, is thereafter reflected there, and returns, how the detected light amount is secured is important. Accordingly, because digital gain adjustment in the image processing does not improve the SN, sensitivity is secured by enhancing the light amount of the light source 102. However, the light amount of acceptable irradiation is limited in consideration of conformity to class 1 of laser safety standards. Thus, instead of increasing the light amount per pulse of the light source 102, the imaging device 100 of this embodiment has a light amount adjustment function for adjusting the light emission frequency of the pulsed light in one frame as illustrated in FIG. 7C. In FIG. 7C, a signal E indicates the waveform of the pulsed light that is emitted from the light source 102. A signal C indicates the waveform in which the surface reflection component I1 and the internally scattered component I2 are combined. A signal D indicates timings of OPEN and CLOSE of the electronic shutter. A signal F indicates timings of charge storage in the charge storage unit. The horizontal axis represents time, and the vertical axis represents the light intensities in the signals C and E, represents the state of OPEN or CLOSE of the electronic shutter in the signal D, and represents a state of OPEN or CLOSE of the charge storage unit in the signal F. The number of pulses of light emission by the light source 102 in one frame is changed, and the irradiation light amount for the object 101 and the detected light amount by the imaging device 100 may thereby be adjusted. The light amount adjustment by changing the number of pulses makes the stability of laser intensity better than a method that changes the current value of a laser diode. Here, the shutter frequency in one frame increases or decreases synchronously with the change in the number of pulses of the light emission. As illustrated in FIG. 7C, as long as times for computation and output processes are secured in one frame, the pulsed light may be increased in the other period than those times. Accordingly, changing the pulsed light per frame means changing the average number of pulsed light that is emitted per unit time.

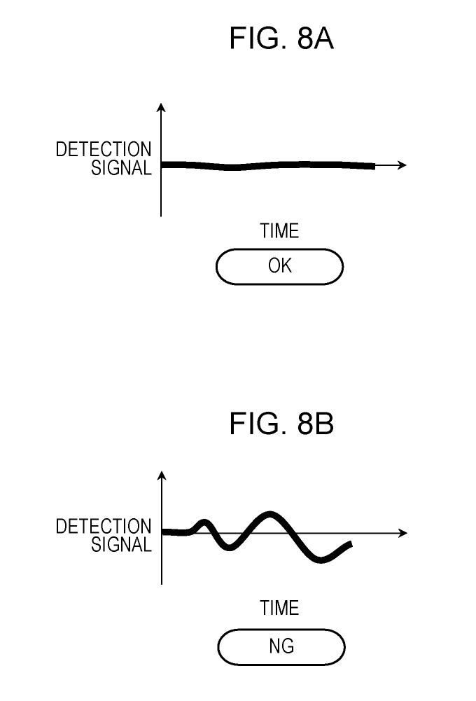

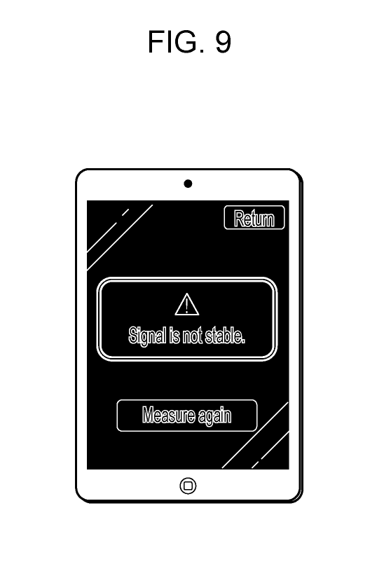

FIG. 8A and FIG. 8B are diagrams that illustrate a function of the signal stability assessment unit 150 of the imaging device 100. The signal stability assessment unit 150 confirms the stability of time-series data of the detection signal in a rest state of the subject. The rest state is a state where the subject thinks about nothing. To induce the rest state of the subject, the subject is caused to keep watching a plain image or to keep watching an image of only a point or a plus sign. Here, as illustrated in FIG. 8A, it is idealistic that the brain blood flow signal of the subject exhibits no increase or decrease and is a regular value. However, depending on the state of the subject, the detection signal is not stable as illustrated in FIG. 8B. One of factors of instability is a case where the mental state of the subject is not a quiet state. In this case, as illustrated in FIG. 9, a fact that the signal is not stable is output on the display 500, and the signal stability is again confirmed after measures such as relaxing the subject and taking time are performed. Further, the detection signal fluctuates in a case where the subject moves during signal stability evaluation or the subject moves his/her eyebrows. The change in the detection signal due to body movement may be determined by calculating oxyhemoglobin and deoxyhemoglobin. Because the measurement is performed contactlessly, the distance between the imaging device 100 and the object 101 fluctuates in a case where the body movement occurs, the irradiation light amount on the object 101 changes, and the light amount that is incident on the object 101 increases or decreases. Accordingly, because the body movement causes the fluctuation in the detection signal, both of oxyhemoglobin and deoxyhemoglobin largely fluctuate in the same direction of the positive or negative direction. Thus, the fluctuations in oxyhemoglobin and deoxyhemoglobin are observed, and the imaging device 100 outputs an error response that instructs the subject not to move in a case where the signal change particular to the body movement is detected. Further, the detection signal may be unstable because the light source 102 is unstable. This is due to a monotonous decrease in the light emission intensity of laser due to a temperature change. In response to that, oxyhemoglobin and deoxyhemoglobin signals seem to be monotonously increasing. Accordingly, based on this monotonous change phenomenon, a determination may be made whether the light source 102 is stable. In this case, the imaging device 100 handles the instability by outputting an instruction for waiting until the light source 102 becomes stable or by conducting a process for calibration correction of the intensity change of the light source 102 due to the temperature. A stability assessment by the signal stability assessment unit 150 enables more accurate measurement in which error factors are reduced or omitted.

In a case where there is no problem in the measurement environment confirmation, light amount adjustment, and detection signal stability confirmation, the final measurement is thereafter started.

### Second Embodiment

In this second embodiment, an imaging device 800 includes an abnormal value assessment unit 810 that detects occurrence of an abnormal value during the measurement. Here, a detailed description about similar contents to the first embodiment in this embodiment will not be made. The abnormal value assessment unit 810 is correspondent to the processor.

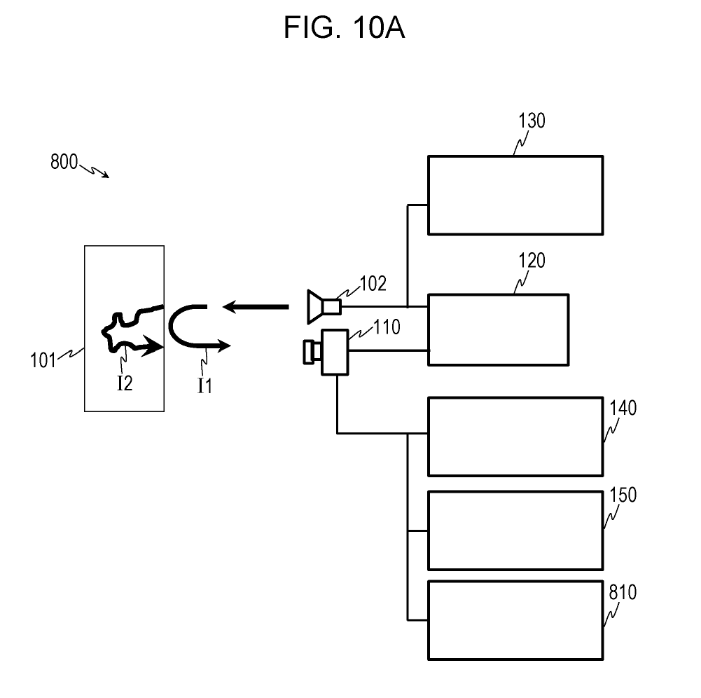

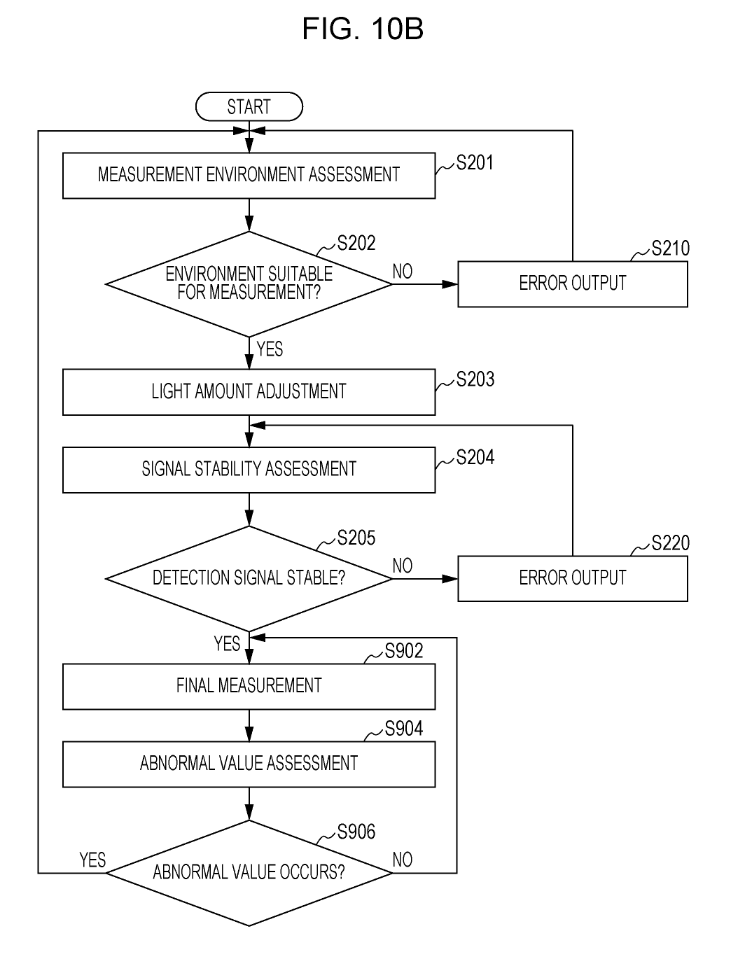

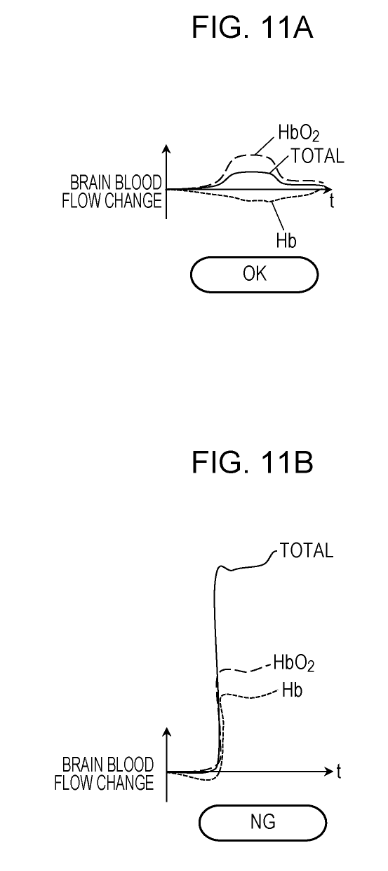

FIG. 10A is a schematic diagram that illustrates the imaging device 800 of the second embodiment and a situation in which the imaging device 800 photographs the object 101. Differently from the first embodiment, the abnormal value assessment unit 810 is added. FIG. 10B is a flowchart that illustrates an action of the imaging device 800 in the second embodiment during the final measurement. In the second embodiment, while the final measurement is conducted (step S902), an assessment about the abnormal value is performed (step S904). In a case where the abnormal value assessment unit 810 assesses the abnormal value as occurring (Yes in step S906), the confirmation of whether or not the environment of the object 101 is in a state suitable for the measurement is conducted (step S201). The abnormal value is for confirming whether an irregular value does not occur to the detection signal during the measurement. For example, the masking object due to hair or the like, the disturbance light, and the body movement are factors of occurrence of the abnormal value. In a case where the masking object such as hair enters during the measurement, the hair absorbs light. Thus, the entrance of the masking object is discriminable because the detection signal excessively lowers and the brain blood flow signal seemingly increases. Further, whether a foreign object enters a camera image of the imaging device 800 is determined by image recognition. Further, in a case where the disturbance light enters, the detected offset component excessively increases. The entrance of the disturbance light is thereby discriminated. Further, in a case where the body movement occurs, the values of oxyhemoglobin (HbO₂) and deoxyhemoglobin (Hb) simultaneously and quickly change. Thus, the occurrence of the body movement may thereby be discriminated. FIG. 11A illustrates time-series data of the brain blood flow change in a case where the abnormal value assessment unit 810 assesses the abnormal value as not occurring. Oxyhemoglobin often increases in a task. However, as for deoxyhemoglobin, a tendency to conversely decrease or slightly increase is often observed. Meanwhile, FIG. 11B illustrates an example where the detection signal largely fluctuates due to the body movement of the subject during the measurement. Because the irradiation light amount increases or decreases on the forehead in the body movement, apparent values of oxyhemoglobin and deoxyhemoglobin together increase or decrease in the same direction. The abnormal value assessment unit 810 displays an error in a case where the signal value exceeds a common blood flow change of human (about 0.1 mM·mm). For example, in a case of 1 mM·mm or more of HbO₂, an abnormal value error is output. Further, because the blood flow change does not occur quickly, in a case where a time-series waveform changes at approximately 90° or a case where a blood flow fluctuation of 0.1 mM·mm or more occurs in one second, the possibility of the abnormal value is high, and a response of the abnormal value error is thus made. Further, whether or not the body movement occurs may be detected by moving body detection image processing computation with image data of the imaging device 800. As the moving body detection, for example, schemes such as optical flow, template matching, block matching, and background subtraction are used.

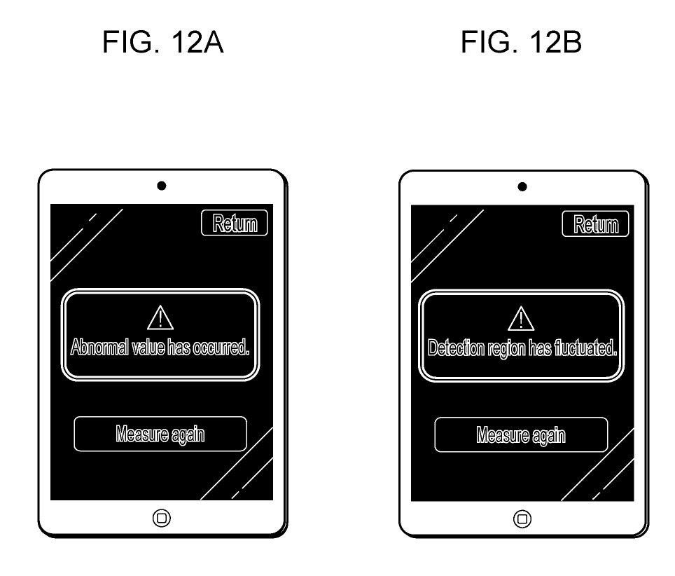

In a case where the abnormal value assessment unit 810 assesses the abnormal value as occurring during the final measurement, as illustrated in FIG. 12A and FIG. 12B, a fact that the abnormal value occurs or displacement of the detection region due to the body movement occurs is output on the display 500. An operator performs a measure against abnormal value factors as necessary and thereafter again conducts the confirmations from the measurement environment confirmation prior to the final measurement, which is described in the first embodiment.

What is claimed is:

1. A device that is used for measurement of an internal portion of an object, the device comprising:
a light source that emits pulsed light with which the object is irradiated;
a light detector that detects light which returns from the object in response to irradiation with the pulsed light; and
a processor, wherein
the processor assesses temporal stability of a light amount of the light which returns from the object and is detected by the light detector.

2. The device according to claim 1, wherein
the processor assesses the temporal stability by determining whether a temporal change of the light amount of the light which returns from the object and is detected by the light detector is within a criteria, and
when it is determined that the temporal change is within the criteria, the processor generates information regarding the internal portion of the object based on a signal from the light detector.

3. The device according to claim 1, wherein
the light detector is an image sensor that converts the light which returns from the object into a signal charge and stores the signal charge, and
the processor assesses the temporal stability by assessing temporal stability of a storage amount of the signal charge in the image sensor.

4. The device according to claim 1, wherein
the processor further, before assessing the temporal stability:
assesses whether an environment of the object is suitable for the measurement of the internal portion of the object, and
adjusts a light amount of the pulsed light.

5. The device according to claim 4, wherein the processor assesses whether the environment of the object is suitable for the measurement by determining whether information regarding the environment of the object is within a criteria.

6. The device according to claim 5, wherein the processor determines whether the information regarding the environment of the object is within the criteria by determining whether a position of a region that is used for the measurement of the internal portion of the object is present in a desired position of the object.

7. The device according to claim 5, wherein the processor determines whether the information regarding the environment of the object is within the criteria by determining whether an amount of a disturbance light that enters the light detector from outside the object is within the criteria.

8. The device according to claim 4, wherein
the processor adjusts the light amount of the pulsed light by adjusting a light emission frequency of the pulsed light per unit time.

9. The device according to claim 3, wherein
the image sensor acquires a first image of the object based on the signal charge, and
the processor further decides a position of a region that is used for the measurement of the internal portion of the object in the first image.

10. The device according to claim 9, wherein
the object is a living body,
the region is an inside of a specific site of the living body, and
the processor further adjusts a size of the region so as to maximize the region in the inside of the specific site.

11. The device according to claim 9, further comprising:
a display, wherein
the display displays the first image and a second image that indicates the region while superimposing the second image on the first image.

12. The device according to claim 11, wherein
the display further displays an additional line for deciding the position of the region while superimposing the additional line on the first image and the second image.

13. The device according to claim 1, wherein
the processor further assesses whether an abnormal value occurs during the measurement of the internal portion of the object.

14. The device according to claim 3, wherein
the image sensor stores the signal charge that corresponds to a component, which is scattered in the internal portion of the object, of the light which returns from the object.

15. The device according to claim 1, wherein
the object is a living body, and
the processor generates information that indicates a blood flow change of the living body based on a signal from the light detector.

16. A method that is used for measurement of an internal portion of an object, the method comprising:
irradiating the object with pulsed light;
detecting light which returns from the object by a light detector in response to irradiation with the pulsed light; and
assessing temporal stability of a light amount of the light which returns from the object and is detected by the light detector.

17. The method according to claim 16, wherein
the light detector is an image sensor that converts the light which returns from the object into a signal charge and stores the signal charge, and
in the assessing,
temporal stability of a storage amount of the signal charge in the image sensor is assessed to assess the temporal stability of the light amount of the light which returns from the object and is detected by the light detector.

18. The method according to claim 16, further comprising:
assessing whether an environment of the object is suitable for the measurement of the internal portion of the object; and
adjusting a light amount of the pulsed light.

19. The method according to claim 16, wherein
the object is a living body, and
the method further includes generating information that indicates a blood flow change of the living body based on a signal from the light detector.
